(19) United States
(12) Patent Application Publication
(10) Pub. No.: US 2018/0110941 A1
(43) Pub. Date: Apr. 26, 2018

(54) PEARL PRECISION MEDICAL DOSING VAPORIZER FULLY INTERCHANGEABLE

(71) Applicants: Brian Smith, Castle Pines, CO (US); John Michael Kasha, Simi Valley, CA (US)

(72) Inventors: Brian Smith, Castle Pines, CO (US); John Michael Kasha, Simi Valley, CA (US)

(21) Appl. No.: 15/476,932

(22) Filed: Mar. 31, 2017

Related U.S. Application Data
(60) Provisional application No. 62/411,509, filed on Oct. 21, 2016.

(51) Int. Cl. A61M 15/00; A61M 11/00

(52) U.S. Cl. CPC A61M 15/004; A61M 2205/3584; A61M 15/0021; A61M 15/0036; A61M 2205/36; A61M 2205/8206; A61M 2205/8237; A61M 2205/50; A61M 2205/3368; A61M 2205/3553; A61M 2205/276; A61M 2205/3358; A61M 2205/609; A61M 2205/6009; A61M 11/00

(57) ABSTRACT

A medicine dosing device for vaporizing a dose of encapsulated liquid medicine inhaling includes a housing with vapor passage between an intake and a mouthpiece. A tank assembly receives the capsule with a closure member pressing the capsule against a needle and spring assembly to puncture the capsule with a hollow needle and press the liquid from the capsule into a tank chamber where is it wicked into the vapor passageway for vaporization. A heater coil between the mouthpiece and the vaporizing wick heats the vaporized medicine before inhalation by the user. A controller having a processor, power management, temperature control, communications and security subsystems enable the medicine dosing device to be enable for use, controlled and monitored remotely with a mobile device.

### CROSS-REFERENCE TO RELATED APPLICATIONS

This non-provisional patent application claims the benefit of U.S. Provisional Patent Application Ser. No. 62/255,209, filed on Nov. 13, 2015, which is incorporated herein by reference in its entirety.

### BACKGROUND OF THE INVENTION

### Technical Field

An apparatus for vaporizing an encapsulated dose of liquid medicine for administration through inhalation and specifically to the device for puncturing the capsule to release and vaporize a dose of the liquid medicine and enabling access, control and monitored of the apparatus remotely with a mobile device.

### Background of the Invention

Various methods of administering drugs are known such as orally, by injection, sublingually, rectally, transdermally and the like. On such method is by inhalation. Drugs administered by inhalation are generally start in a liquid form with the liquid being atomized into smaller droplets so that the drugs can pass into the lungs. How deeply into the lungs they go depends on the size of the droplets. Smaller droplets go deeper, which increases the amount of drug absorbed. Inside the lungs, the drugs are absorbed directly into the bloodstream. Inhaled drugs are also more effectively localized to the target organ, which generally allows for a lower dose than is necessary with systemic delivery (oral or injection) thus resulting in fewer and less severe adverse effects.

While inhalation is an effective delivery method, care in administration is important particularly when it is important that a person receives the right amount of drug at a proper rate. This is not as critical for substances such as nicotine or cannabis where dosages can vary or can be user limited. Nevertheless, as the medical uses of cannabis expand, the importance and advantages of the application of more precise dosages increases.

The present invention is an medication application apparatus and system that enables increased precision in the application by providing defined dosages of medication in a generally spherical medication containing capsule that is punctured or ruptured to release the mediation dosage and then vaporizing the mediation for inhalation where the apparatus control for the rate of application and then controls use using a remote communication device such as a smart phone to monitor, record, control access, and control operation parameters.

### SUMMARY OF THE INVENTION

A medicine dosing device for vaporizing a liquid medicine delivered to the device from a capsule containing a defined dosage of the medication in liquid form includes a housing, a tank assembly for being removably inserted into the housing, a heater coil for heating a vapor formed from the liquid mediation and an electronic controller for control operation of the medicine dosing device. The electronic controller includes a processor subsystem which has one or more microprocessors to control access, operation and monitoring of the device; a power management subsystem for providing power to the processor subsystem and other electronic components of the dosing device; a temperature control subsystem for controlling the temperature of the vapor to be inhaled by the user; an access control subsystem for limiting use of the dosing device to predefined users; an elevation sensor to provide pressure data to the processor for enable adjustment of operational parameters in response to atmospheric pressure; and a communication subsystem for providing a communication link between the processor and a remote mobile device.

The power management subsystem is couple to power the various electronic components of the system and includes a battery, a charging coil for receiving energy wirelessly from a remote energy source, charging coil coupled to a battery charger for providing energy from the remote energy source for charging the battery.

The temperature control subsystem includes a temperature sensor positioned to sense the temperature of the vapor in a vapor passageway extending through the housing between an air input on one end and a mouthpiece on the opposite end. The sensor is position in the vapor passageway between the heater coil and the mouth piece to insure the temperature is of the heated vapor. The temperature sensor generates a temperature signal which is coupled to the processor subsystem. The processor subsystem generating a heater coil control signal which is coupled to the heater coil to control the heating of the heater coil in response to the temperature signal to maintain the temperature of the vapor in the vapor passageway at the preselected temperature.

The access control subsystem includes a biometric sensor such as a finger print scanner or a face recognition sensor or other sensor of a user biometric. Also included is a memory for storing biometric characteristic data derived from an authorized user. The memory may be incorporated in the processor subsystem. The biometric data of an authorized user may be obtained, for example, from a remote communication device such as a smart phone programmed to read the selected biometer parameter and then communicate that information for storage in the memory of the processor subsystem. The processor subsystem is then couple to the biometric sensor for receiving biometric data from a person seeking to use the medicine dosing device where that received data is compared against the stored biometric data with operational use of the medicine dosing device allowed only when the sensed biometric data matches the stored biometric data. If there is a match, the processor subsystem enables, for example, heating of the coil or the functioning of other electronic components of the medicine dosing device.

### BRIEF DESCRIPTION OF THE DRAWINGS

For a more complete understanding of the present invention and the advantages thereof, reference is made to the following description taken in conjunction the accompany drawings wherein like reference numerals represent like parts, in which:

FIG. 1 is perspective front view showing a medication dosing vaporizer device;

FIG. 2 is perspective rear view showing a medication dosing vaporizer device;

FIG. 3 is as exploded perspective view of the medication dosing vaporizer device;

FIG. 4 is front perspective view of at tank assembly of the medication dosing vaporizer device;

FIG. 5 is a side view of the tank assembly of the medication dosing vaporizer device;

FIG. 6 is a top view of the tank assemble of the medication dosing vaporizer device;

FIG. 7 is a block diagram showing the electronic controller of the medication dosing vaporizer device.

### DETAILED DESCRIPTION OF THE INVENTION

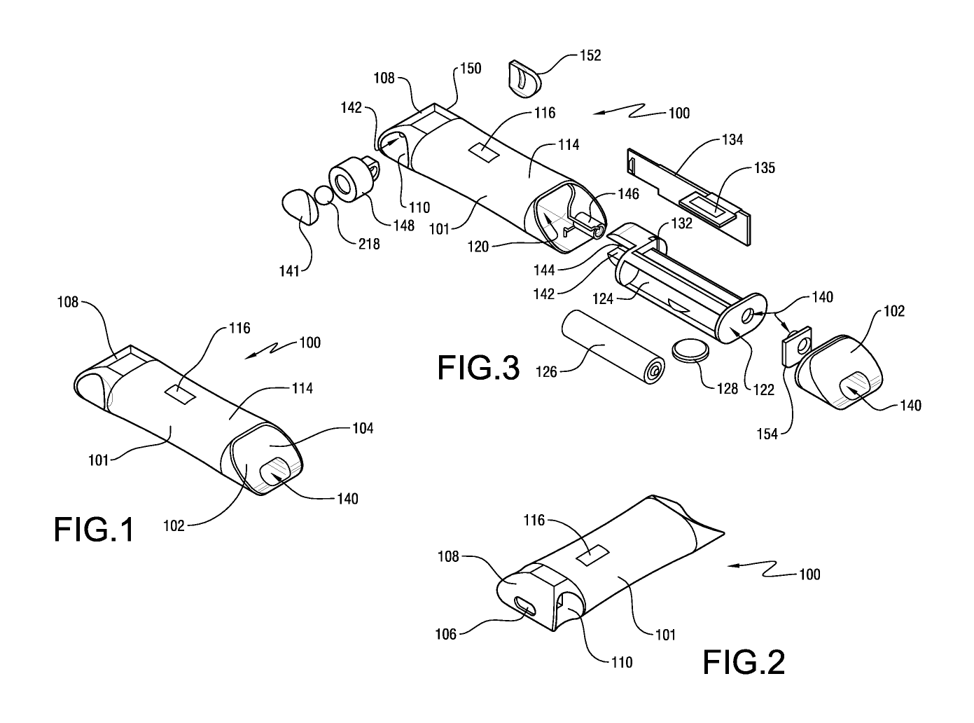

Referring to FIGS. 1 and 2, a medicine dosing vaporizer device 100 is illustrated having a housing 101 with a mouth piece 102 at a first end 104, an air intake opening 106 at a second end 108 and a capsule or pearl receiving opening 110 proximate to the second end 108. A finger scanner (touch pad) 116 optionally position at an intermediate position on the top 114 of the housing 101 for enabling a user to be able to use the medicine dosing device 100. The finger print scanner 116 allows for controlling access of use to those persons whose finger print characteristic matches a previously stored finger print characteristics as will be hereafter described.

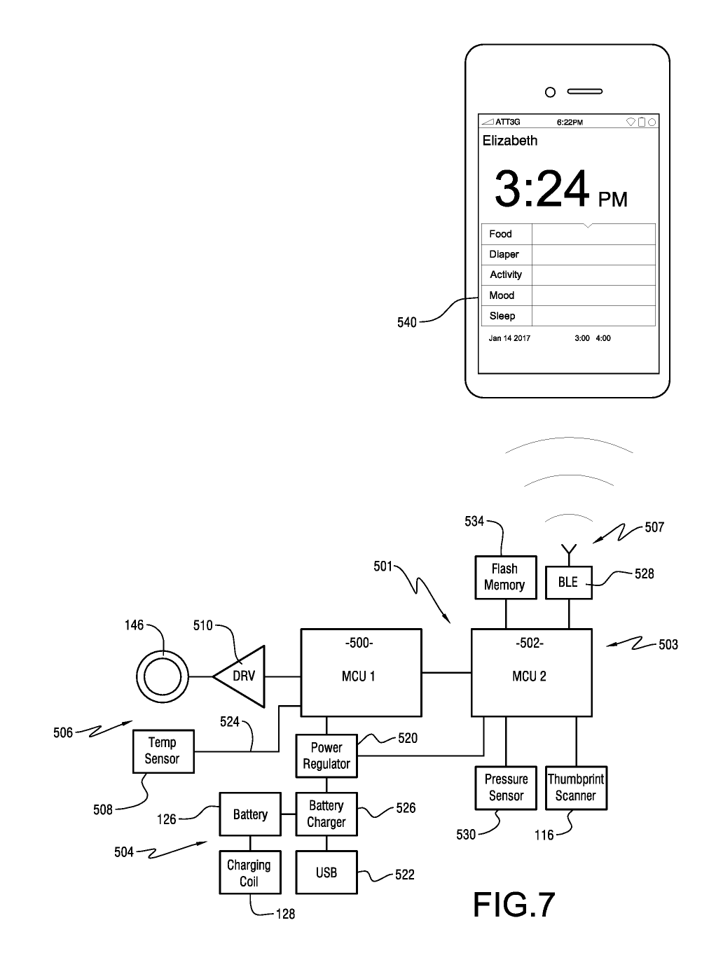

A pictorial view of the medicine dosing vaporizer 100 of FIGS. 1 and 2 is shown in FIG. 3 where the housing 101 has an axial chamber 120 defining an interior space and the mouth piece 104 is separate from the housing 101. A removable insert 122 is configured to fit into the axial chamber 120. The removable insert 122 includes a battery receiving recess 124 for receiving a battery 126 which preferably is rechargeable. Juxtaposed adjacent to the bottom inside of the housing 101 is a charging coil 128 (which may be attached to the removable insert 122) electrically coupled to the battery 126 through a battery charger 526 (FIG. 7) for recharging the battery 126 when the charging coil 128 is placed in power transferring relationship to an extrinsic power coil (not shown). The removable insert 122 also has a support slot 132 to which a support member 134 is affixed. Electronic circuits 135 representing selected functionality of an electronic controller 503 (described in conjunction with FIG. 7) are mounted on the support member 134.

A vapor passageway 140 extends from the air intake opening 106 at the air intake (second) end 108 of the housing 101, through the removable insert 122 and out through the mouth piece 102 at the first end 104. The removeable insert 122 further includes a tank assembly receiving recess 142 and a heater recess 144 extending from the tank assembly receiving recess 142 to intersect and define a part of the vapor passageway 140 which in the illustrated embodiment is proximate the second end 108 of the housing 101 when the removable insert 122 is in the axial chamber 120 of the housing 101. A heater coil 146 is positioned in the heater recess 144 and the tank assembly receiving recess 142 is juxtaposed adjacent the capsule receiving opening 110 when the removable insert 122 is fully inserted into the housing 101. Also interposed in the vapor passageway between the removable insert 122 and the mouth piece 102 is an inner nozzle 154.

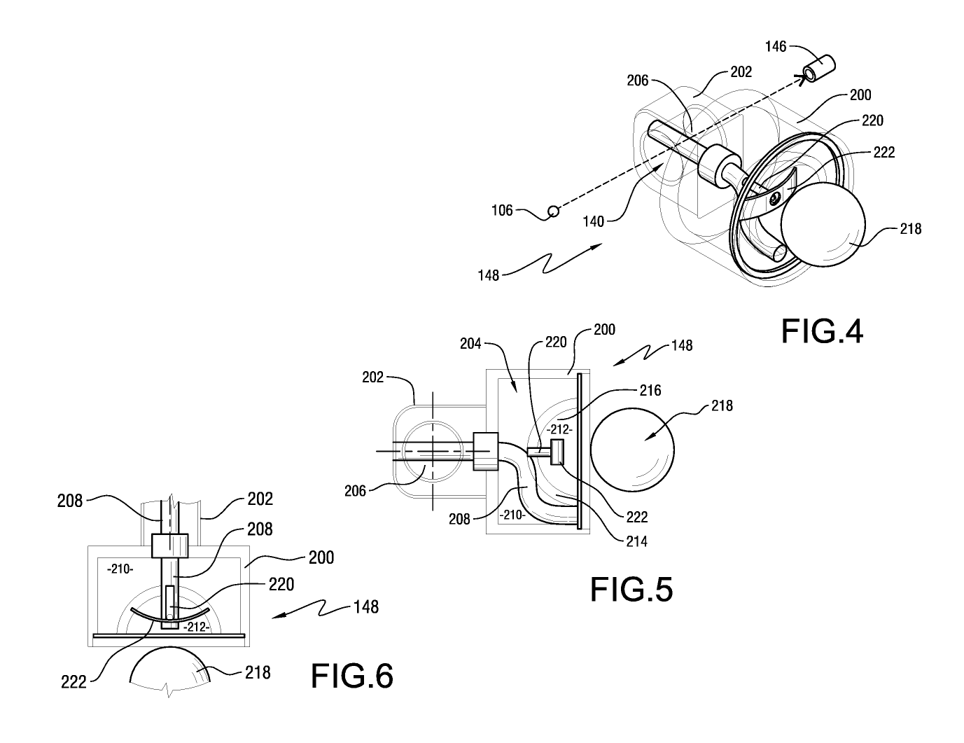

A tank assembly 148 is removably inserted through the capsule receiving opening 110 in the housing 101 into the tank assembly receiving recess 142 of the removable insert 122 with a closure member 141 positioned to engage a medication containing capsule (pearl) 218 (FIGS. 4, 5 and 6) and press the pearl into the tank assemble 148 as will be hereafter described in conjunction with FIGS. 4, 5, and 6. Juxtaposed opposite the capsule receiving opening 110 is a USB opening 150 in the housing 101 for locating a USB electrical connector (not shown) as an alternative wired connection to, for example, powering or recharging the medicine dosing vaporizer 100. A UCB closure member 152 may be removably positioned over the UCB opening 150.

Referring to FIGS. 4, 5 and 6 in conjunction with FIG. 3, the tank assembly 148 is removably positioned in the tank assembly receiving recess 142 adjacent the capsule receiving opening 110. The tank assembly 148 has a tank member 200 and a passage member 202 which extending therefrom to intersect the vapor passageway 140. The tank member 200 defines a tank chamber 204 and the passage member 202 defines a passage chamber 206 which is part of the vapor passageway 140 located between the air intake end 108 and the heater recess 144 in which the heater coil 146 is positioned. The tank chamber 204 is divided into a liquid receiving chamber 210 and a capsule receiving chamber 212 by a concave wall 214. A wick 208, which may be cotton or other wicking material, extends between the liquid receiving chamber 210 and the passage chamber 206 to provide a pathway for liquid injected or otherwise inserted into the tank chamber 204 to be wicked from the liquid receiving chamber 210 to the passage chamber 206 where the liquid vaporizes as air is drawn from the air intake opening 106 through the passage chamber 206 to the mouth piece 104 in response to negative pressure provided by a user inhaling through the mouth piece 104. More specifically, the user by exerting a negative pressure on the mouth piece causes air to be drawn through the air intake opening 108 into the passage chamber 206 where the air is mixed with the vaporized liquid from the wick, after which the vapor is heated by the heater coil 146 in the heater recess 144, and is drawn along the remainder of the vapor passageway 140 to the mouth piece 104 for administration to the user.

The tank chamber 204 is divided into a liquid receiving chamber 210 and a capsule receiving chamber 212 by a wall 214. The wall 214 has an interior concave surface 216 toward which a generally spherical capsule such as capsule 218 may be inserted. A capsule puncturing device such as a hollow needle 220 is fixed to extend through wall 214 to provide a path for transferring the liquid from the capsule 218 to the liquid receiving chamber 210. A spring 222 is positioned in the capsule receiving chamber 212 to press outwardly against the capsule 218.

In operation, a capsule such as the capsule 218 is inserted into the capsule receiving chamber 212 in puncturing relationship to the pointed end of the hollow needle 220. The closure member 141 (FIG. 3) is positioned next to the capsule 218 and is pressed to a closed position over the capsule receiving opening 110. As the closure member 141 is pressed into this closed position, the capsule 218 is pressed against the sharp end of the needle 220 which punctures the capsule 218 creating a path for the liquid in the capsule to from the capsule 218 through the hollow center of the needle 220 into the liquid receiving chamber 210. As the closure member 141 also presses the capsule 218 against the spring 222 which together with closure member 141 causes the liquid in the capsule to be squeezed from the capsule 218, through the hollow needle 220 and into the liquid receiving chamber 210. The liquid is then wicked from the liquid receiving chamber 210 into the passage chamber 206 where the liquid vaporizes and is drawn through the vapor passageway 140 as above described. When the closure member 141 is removed from its closure position, the spring 222 pressing against the capsule 218 causes the capsule 218 to be pushed off the needle 220 and expelled out of the capsule receiving chamber 212.

The liquid from the capsule 222 may be transferred into the tank chamber 204 in other ways as well such as direct injection into the liquid receiving chamber. The dosage in the capsule can be set by varying the size of the capsule, the quantity of medicine in the capsule, the concentration of the medicine, the rate of wicking and similar factors. The capsule 210 is preferably made of a water insoluble material which encases the predefined dosage of medicine.

A block diagram of the electronic controller 503 is shown in FIG. 7 and includes a processor subsystem 501 which can be a single processor or which in the embodiment shown includes a first microprocessor 500 and a second microprocessor 502. The microprocessors 500 and 502 may be electronic circuits mounted on the support member 134 (FIG. 3). The first microprocessor 500 is coupled to a power management subsystem 504, and a temperature control subsystem 506 and a communication subsystem 507 for providing control and communication between the electronic controller 503 and a mobile device 540.

The power management subsystem 504 includes the battery charger 526 coupled to a power regulator 520 which is coupled for suppling power to the first and second microprocessors 500 and 502; the charging coil 128 which is coupled to the battery 126 which is also coupled to the battery charger 526; and a USB connector 522 (positioned in the USB opening 150 in FIG. 3) and to the battery charger 526. In operation, the battery can be charge through either the charging coil 128 or an extrinsic power source coupled through the USB connector 522.

The temperature control system 506 includes a temperature sensor 508 coupled to provide temperature information to the first microprocessor 500; the first microprocessor 500; the heater coil 146 (also shown in FIG. 3); and a driver circuit 510 coupled between the first microprocessor 500 and the heater coil 146. In operation, the temperature sensor 508 which is positioned near the heater 146 between the heater 146 and mouthpiece to sense the temperature of the vapor heated by the heater coil 146, provides a temperature signal 524 from the temperature sensor 508 to the first microprocessor 500 where is it compared to a desired temperature value stored in a memory such as memory 534. The difference between the measured temperature and the desired temperature values is used by the first microprocessor 500 to generate a signal which is coupled to the driver 510 to either increase or decrease the power supplied to heat the coil 146 as in a conventional feedback control system.

The communication subsystem 507 uses the second microprocessor 502 to manage the wireless connectivity to a remote mobile device 540 through, for example, a BLE (Bluetooth Low Energy) transmitter 528 so that a user can set and monitor functional parameters of the medicine dosing vaporizing device remotely using the mobile device 540.

As shown in FIG. 7, an altitude or pressure sensor 530 provides a measure of altitude which is coupled to the second microprocessor 502 which provides that information to the remote mobile device 540 through the BLE transmitter 528 where altitude/pressure can be displayed.

A security subsystem is also coupled to the second microprocessor 502 and includes the finger print scanner 116 and the memory 534, both coupled to the second microprocessor 502. In order control who used the medicine dosing device 100, the second microprocessor 502 is programmed to enable, for example, the heater coil 146 only when finger print information from the finger print scanner 116 matches finger print information stored in the memory 534. Specifically, an authorized user first runs a mobile app on the mobile device 540 to remotely scan their finger or thumb print. That data is then communicated via the BLE 528 to the second microprocessor 502 which stores the fingerprint information in the memory 534. Multiple users can have their fingers scanned and the finger print information stored in the memory 534 through the remote mobile device 540 using the app. The electronic controller 503 then allows the medicine dosing system 100 to be used only if the finger print scanned by the scanner 116 matches one of the finger prints stored in the memory 534.

As will be appreciated, any other desired parameter or control feature in addition to temperature, pressure, power management and access security can be incorporated and managed and monitored through the remote mobile device using the communications subsystem 507 without departing from the invention in its broadest aspects.

The apparatus, devices and systems and their interconnections describe above and shown in the drawing disclose an illustrative embodiment only. Other embodiments, details, changes, substitutions, and alterations can be made and such will be readily ascertainable by one skilled in the art without departing from the spirit and scope of the invention as defined by the following claims.

What is claimed is:

1. A medicine dosing device for vaporizing a liquid medicine contained in a capsule and delivering a selected dosage of the vaporized liquid medicine to a user through inhalation comprising:
A housing having a first end with a mouth piece, an air intake end opposite the first end, a vapor passage extending from the air intake end to the mouth piece, and a capsule receiving opening;
A tank assembly positioned in housing proximate to the capsule receiving opening comprising:
a tank member positioned proximate to the capsule receiving opening,
a passage member extending from the tank member, the passage member having a passage chamber positioned for being a part of the vapor passageway adjacent to the air intake end of the housing,
a wick extending between the tank member and the passage chamber for wicking liquid in the tank member to the passage chamber to form a vapor of the wicked liquid in the vapor passageway;
a capsule puncturing device positioned in the tank member proximate to the capsule receiving opening;
a spring positioned in the tank member for pressing the capsule positioned in the tank member;
a closure member for selectively moving to a closed position pressing the capsule into puncturing relationship with the puncturing device and further against the spring for releasing the liquid in the capsule into the tank member; and
A heater coil mounted in the vapor passageway to heat the vapor of the wicked liquid to a predetermined temperature.

2. The medicine dosing device of claim 1 further comprising a dividing wall in the tank member to divide the tank member into a liquid receiving chamber and a capsule receiving chamber with the capsule puncturing device extending through the dividing wall and the spring positioned in the capsule receiving chamber.

3. The medicine dosing device of claim 2 wherein the dividing wall is a curved wall.

4. The medicine dosing device of claim 1 further comprising:
A battery coupled to the heater coil, the battery energizing the heater coil to heat the vapor in the vapor passageway.

5. The medicine dosing device of claim 4 wherein the battery is rechargeable.

6. The medicine dosing device of claim 4 further comprising:
A battery charger coupled for charging the battery; and
A charging coil for receiving energy wirelessly from a remote energy source, the coil attached to the housing and coupled to the battery charger for providing energy from the remote energy source for charging the battery.

7. The medicine dosing device of claim 1 further comprising an electronic controller for controlling the medicine dosing device comprising:
A processor subsystem;
A power management subsystem couple to the processor subsystem for providing power to the medicine dosing device;
A temperature control subsystem coupled to the processor subsystem for controlling the temperature of the vapor in the vapor passageway by controlling the energy that heats the heater coil;
Communication subsystem for providing a communication link between the processor subsystem and a remote mobile device; and
A security subsystem couple to the processor subsystem for limiting access to use of the medicine dosing device.

8. The medicine dosing device of claim 7 wherein the temperature control subsystem comprises:
a temperature sensor positioned to sense the temperature of the vapor in the vapor passageway, generate a temperature signal therefrom and provide the temperature signal to the processor subsystem, the processor subsystem generating a heater control signal couple to the heater coil to control the heating of the heater coil in response to the temperature signal to maintain the temperature of the vapor in the vapor passageway at the preselected temperature.

9. The medicine dosing device of claim 7 where in the power management subsystem comprises:
A battery coupled to provide electrical power to the medicine dosing device;
A battery charger coupled for charging the battery;
A charging coil coupled to the battery charger for receiving energy wirelessly from a remote energy source for providing energy from the remote energy source for charging the battery.

10. The medicine dosing device of claim 7 wherein the electronic controller further comprises:
An elevation sensor providing a pressure signal to the processor subsystem, the processor subsystem controlling the operation of the medicine dosing device in response to the pressure signal.

11. The medicine dosing device of claim 7 wherein the access control subsystem comprises:
a biometric sensor for sensing a biometric characteristic of a person seeking to use the medicine dosing device;
a memory in the for storing biometric characteristic data of an authorized user, and
the processor subsystem couple for comparing the sensed biometric characteristic against the stored biometric characteristic and granting operational use of the medicine dosing device only when the sensed biometric characteristic matches the stored biometric characteristic.

12. The medicine dosing device of claim 11 wherein the biometric sensor is a finger print scanner.

13. The medicine dosing device of claim 7 wherein the communication subsystem is coupled to the processor subsystem using a Bluetooth Low Energy device for transmitting and receiving operation control and status information between the medicine dosing device and the remote mobile device.

14. A medicine dosing device for vaporizing a liquid medicine contained in a capsule and delivering a selected dosage of the vaporized liquid medicine to a user through inhalation comprising:
A housing having a first end with a mouth piece; and
A tank assembly positioned in the housing in vapor providing relationship with the mouth piece, the tank assembly comprising:
a tank member having a capsule receiving opening,
a capsule puncturing device positioned in the tank member proximate to the capsule receiving opening;
a closure member coupled to the tank member for selectively moving to a closed position over the capsule receiving opening for pressing the capsule into puncturing relationship with the puncturing device and transferring the liquid from the capsule into the tank member; and
a heater mounted in the tank member for heating the liquid to form a vapor and directed that vapor to the mouth piece.

15. The medicine dosing device of claim 14 further comprising a dividing wall in the tank member to divide the tank member into a liquid receiving chamber and a capsule receiving chamber with the capsule puncturing device extending through the dividing wall from the liquid receiving chamber and the capsule receiving chamber.

16. The medicine dosing device of claim 15 wherein the dividing wall is a curved wall.

17. The medicine dosing device of claim 14 further comprising:
A battery coupled to the heater, the battery energizing the heater to heat the liquid in the tank member.

18. The medicine dosing device of claim 17 wherein the battery is rechargeable.

19. The medicine dosing device of claim 17 further comprising:
A battery charger coupled for charging the battery; and
A charging coil for receiving energy wirelessly from a remote energy source, the coil attached to the housing and coupled to the battery charger for providing energy from the remote energy source for charging the battery.

20. The medicine dosing device of claim 14 further comprising an electronic controller for controlling the medicine dosing device comprising:
A processor subsystem;
A power management subsystem couple to the processor subsystem for providing power to the medicine dosing device;
A temperature control subsystem coupled to the processor subsystem for controlling the temperature of the vapor provided to the mouthpiece by controlling the energy that heats the heater coil;
Communication subsystem for providing a communication link between the processor subsystem and a remote mobile device; and
A security subsystem couple to the processor subsystem for limiting access to use of the medicine dosing device.

21. The medicine dosing device of claim 20 wherein the temperature control subsystem comprises:
a temperature sensor positioned to sense the temperature of the vapor in the vapor passageway, generate a temperature signal therefrom and provide the temperature signal to the processor subsystem, the processor subsystem generating a heater control signal couple to the heater to control the heating of the heater in response to the temperature signal to maintain the temperature of the vapor at the preselected temperature.

22. The medicine dosing device of claim 20 where in the power management subsystem comprises:
A battery coupled to provide electrical power to the medicine dosing device;
A battery charger coupled for charging the battery;
A charging coil coupled to the battery charger for receiving energy wirelessly from a remote energy source for providing energy from the remote energy source for charging the battery.

23. The medicine dosing device of claim 20 wherein the electronic controller further comprises:
An elevation sensor providing a pressure signal to the processor subsystem, the processor subsystem controlling the operation of the medicine dosing device in response to the pressure signal.

24. The medicine dosing device of claim 20 wherein the access control subsystem comprises:
a biometric sensor for sensing a biometric characteristic of a person seeking to use the medicine dosing device;
a memory in the for storing biometric characteristic data of an authorized user, and
the processor subsystem couple for comparing the sensed biometric characteristic against the stored biometric characteristic and granting operational use of the medicine dosing device only when the sensed biometric characteristic matches the stored biometric characteristic.

25. The medicine dosing device of claim 24 wherein the biometric sensor is a finger print scanner.

26. A vaporizer apparatus for vaporizing and delivering a selected dosage of liquid medicine comprising:
A housing having an axial chamber therein with an axial opening at a first housing end and a tank assembly opening, an air intake opening and a USB connector port at a second housing end opposite the first housing end;
a mouth piece connected to the first housing end;
a removeable insert positioned in the axial chamber, the insert having a battery support recess, a controller attachment mount, a tank assembly receiving recess for alignment adjacent the tank assembly opening, a UCB connector recess aligned adjacent the USB connector port, a heater recess and a vapor passageway extending axially through the insert for providing a vapor passageway from the air intake opening, through the tank assembly receiving recess, the heater recess and the mouth piece;
a tank assembly positioned in the tank receiving recess comprising:
a tank member having a tank chamber with a capsule receiving chamber;
a passage member extending from the tank chamber, the passage member having a passage chamber therethrough defining a part of the vapor passageway between the air intake opening and the heater recess;
a wick extending between the tank chamber and the passage chamber for transferring a liquid medicine dose from the tank chamber to the passage chamber to provide a vapor of the liquid medicine in the passage chamber;
a hollow capsule puncturing member positioned between the capsule receiving chamber to the tank member to puncture a capsule having therein a dosage of liquid medicine pressed into the capsule receiving chamber for transferring the liquid from the capsule to the tank member;
a closure member positioned for closing capsule receiving chamber and pressing the capsule against the capsule puncturing member to release the dose of liquid medicine from the capsule into the tank chamber;
a spring position in the tank chamber to press against a capsule position in the capsule receiving chamber to urge the liquid medicine from the capsule to tank member when the closure member is urged to a closed position and ejecting the capsule from the capsule receiving chamber when the closure member is moved to an open position;
a battery mounted in the battery support chamber;
controller attached to the controller attachment mount and coupled to be powered by the battery and including a communications subsystem coupled to controller for providing a communication link between the controller and a remote mobile device;
a charging coil attached in the hollow chamber adjacent the bottom of the housing and coupled for wirelessly charging the battery;
a heater coil mounted in the heater recess in the vapor passageway of the support member for heating vapor in the vapor passageway,
a temperature sensor positioned for sensing the temperature of the vapor in the vapor passageway, the heater coil and temperature sensor couple to the controller for controlling the heating of the heater coil in response to the sensed temperature from the temperature sensor.
